(19) United States
(12) Patent Application Publication
(10) Pub. No.: US 2025/0290911 A1
(43) Pub. Date: Sep. 18, 2025

(54) SYSTEMS AND METHODS FOR AUTOMATED GROUNDWATER DATA COLLECTION BACKGROUND

(71) Applicant: DuBois Joseph Ferguson, Houston, TX (US)

(72) Inventor: DuBois Joseph Ferguson, Houston, TX (US)

(21) Appl. No.: 18/604,135

(22) Filed: Mar. 13, 2024

(51) Int. Cl. G01N 33/18; G01N 21/25

(52) U.S. Cl. CPC G01N 33/18; G01N 21/255; G01N 2201/0216; G01N 2201/062

(57) ABSTRACT

Systems and methods are provided for automated groundwater data collection. Particularly, the systems and methods described herein allow for the automated analysis of the groundwater samples and collection of the resulting data using fluorescence analysis. The system may include one or more groundwater collection and analysis apparatuses provided at various wells including groundwater that is desired to be sampled. A UAV equipped with a light source navigates to the location of the wells and emits light at particular wavelengths into the groundwater collection and analysis apparatuses. The groundwater collection and analysis apparatuses hold samples of the groundwater in the well and perform analyses of the contents of the groundwater using the emitted light. The resulting data is then provided to the UAV and/or a remote system for storage.

### BACKGROUND

Conventionally, groundwater sampling (for example, to identify the contents of the groundwater, including any contaminants) is performed by a field crew that traverses to the location of a well including the groundwater to be analyzed. Once at the location, the field crew manually collects groundwater samples that require subsequent off-site laboratory analysis. The process is time-consuming and often requires the members of the field crew to travel into environments that are difficult or otherwise undesirable to traverse, such as swamps, for example. For many environmental remediation projects, groundwater sampling and analysis represent the greatest costs incurred. Depending on the specific regulatory requirements, the sampling frequency can be on a quarterly, semi-annual, or annual basis. Due to the lengthy clean up times involved, typically associated with the remediation of impacted groundwater aquifers, the post-closure monitoring and maintenance period is often a minimum of 30 years.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. In the drawings, the left-most digit(s) of a reference numeral may identify the drawing in which the reference numeral first appears. The use of the same reference numerals indicates similar, but not necessarily the same or identical components. However, different reference numerals may be used to identify similar components as well. Various embodiments may utilize elements or components other than those illustrated in the drawings, and some elements and/or components may not be present in various embodiments. The use of singular terminology to describe a component or element may depending on the context, encompass a plural number of such components or elements and vice versa.

FIG. 1 illustrates a system for groundwater data collection, in accordance with one or more example embodiments of the disclosure.

FIG. 2 illustrates another system for groundwater data collection, in accordance with one or more example embodiments of the disclosure.

FIGS. 3A-3C illustrates additional components of a system for groundwater data collection, in accordance with one or more example embodiments of the disclosure.

FIG. 4 illustrates another system for groundwater data collection, in accordance with one or more example embodiments of the disclosure.

FIG. 5 illustrates another system for groundwater data collection, in accordance with one or more example embodiments of the disclosure.

FIG. 6 illustrates a method for groundwater data collection, in accordance with one or more example embodiments of the disclosure.

FIG. 7 illustrates another example system for groundwater data collection, in accordance with one or more example embodiments of the disclosure.

FIG. 8 illustrates a computing device, in accordance with one or more example embodiments of the disclosure.

### DETAILED DESCRIPTION

This disclosure relates to, among other things, systems and methods for automated groundwater data collection. In contrast with conventional groundwater analysis, which requires a field crew to manually traverse to the locations of the wells including the groundwater samples to be analyzed, the systems and methods described herein allow for the automated analysis of the groundwater samples and collection of the resulting data using fluorescence analysis.

Fluorescence analysis is a type of analysis that may be used to analyze dissolved organic matter within water systems (for example, groundwater samples from a well as described herein). As a non-limiting example, a fluorescence analysis may include a two-dimensional (2D) fluorescence analysis technique, such as fluorescence excitation spectroscopy, fluorescence emission spectroscopy, and synchronous fluorescence spectroscopy. A 2D fluorescence spectrum represents the relationship between the fluorescence intensity and excitation wavelength or emission wavelength. 2D spectra are simple and intuitive with the x-axis representing the excitation wavelength or emission wavelength, and the y-axis representing the relative fluorescence intensity. This fluorescence spectrum may be determined, for example, using a spectrometer that is provided in the groundwater collection and analysis apparatus. From a 2D fluorescence spectrum, a system may intuitively obtain the number and position of fluorescence peaks. These peaks can then be used to identify fluorescent substances or as the basis for selecting appropriate excitation and emission wavelengths for the fluorescence analysis of substances. Oher spectroscopy techniques such as a cavity ring down spectroscopy (CRDS) system or Fourier-transform infrared (FTIR) spectroscopy could also be used. These are merely examples of types of fluorescence analyses and any other suitable techniques may also be used.

To facilitate this automated process, a UAV may be instructed to navigate to the locations of the wells from which the analyses are to be performed. The UAV may be equipped with a light source that is configured to emit light at particular wavelength(s) toward a groundwater collection and analysis apparatus provided on and/or within each of the wells. For example, the light source may be a continuous-wave (CW) diode laser that is mounted to the UAV with an output power of 1 W at wavelengths ranging between 200 to 800 nm for the detection of groundwater contaminants. This is merely one example of a specific light source and any other type of light source configured to emit light at varying wavelengths and power levels may also be used.

The groundwater collection and analysis apparatus may be configured to be provided on and/or within a standard two-inch diameter Polyvinylchloride (PVC) construction monitoring well, however, the groundwater collection and analysis apparatus may also be configured to fit on and/or within any other size of well. The groundwater collection and analysis apparatus may include a cuvette that holds a sample of groundwater from the well.

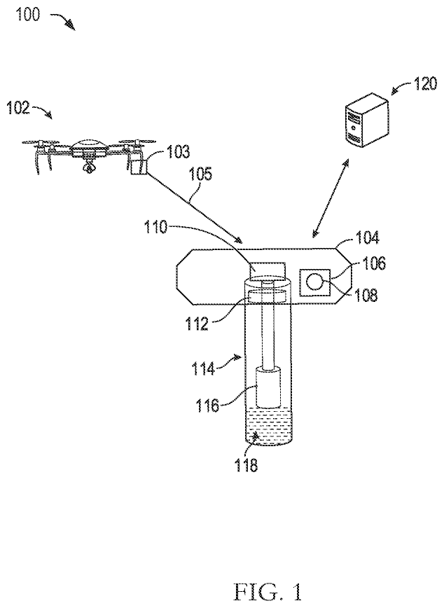

When the groundwater collection and analysis apparatus receives the light emitted by the UAV, the light is directed to the cuvette to induce the fluorescence of target chemical compounds contained in a groundwater sample. The associated emission spectra is captured and analyzed using the spectrometer. The data produced by the spectrometer may be used to determine the contents of the groundwater sample. For example, the data may be used to determine specific types of contaminants that are found within the groundwater sample. This data may then be transmitted to the UAV and/or to a remote system (such as a remote server) for storage (and/or the data may be stored locally in data storage of the groundwater collection and analysis apparatus. For example, the groundwater collection and analysis apparatus may include a microprocessor with communication capabilities or a standalone transceiver. This data transmission may be performed using any wired or wireless communication protocol. Additional details about components that may be included within the groundwater collection and analysis apparatus are described in greater detail with respect to at least FIGS. 1-3C.

The groundwater collection and analysis apparatus may also include various components used to provide a sample of the groundwater in the well into the cuvette for analysis. This may be accomplished in a number of different ways. As a first example, the groundwater collection and analysis apparatus may include one or more pumps that may be used to pump a sample of groundwater from the well into the cuvette. In some instances, the pump may be a submersible pump. Additionally, multiple pumps may be used. For example, one pump may be used to pump the groundwater from the well into a groundwater reservoir for temporary storage and another pump may then pump the water from the groundwater reservoir into the cuvette. However, in some instances, only one pump may be used or more than two pumps may be used as well. Additionally, the sample may be pumped directly into the cuvette rather than being pumped into the groundwater reservoir (that is, the groundwater reservoir may not be required). The cuvette may also include a drain tube that is used to return the sample of groundwater back into the groundwater at the bottom of the well. Thus, the groundwater sample may only need to be held temporarily within the cuvette.

Another option is to use a valve to trap vapor that is naturally produced within the well. This vapor may naturally result from changes in barometric pressure within the well (Henry's law states that the amount of dissolved gas in a liquid is directly proportional to its partial pressure above the liquid). For example, the valve may be a one-way check valve, however, other types of valves may be used as well. In this manner, the analysis may also be performed using a vapor sample rather than the use of a liquid groundwater sample. In embodiments, the vapor may also be condensed within the groundwater collection and analysis apparatus such that the vapor sample may be converted into a liquid groundwater sample for analysis.

In embodiments, a light source may also be provided on or within the well itself (in addition to, or alternatively to, the light source being provided on the UAV). For example, UV-C LEDs (or any other type of light source) may be provided on and/or within the groundwater collection and analysis apparatus or as a standalone component. In such embodiments, rather than requiring the UAV to navigate to the location of the well and emit light toward the groundwater collection and analysis apparatus, the light source provided at the well itself may emit the light that is used for the analysis of the groundwater sample. In such embodiments, a controller (such as the aforementioned microprocessor or any other device including processing capabilities) included within the groundwater collection and analysis apparatus may instruct the light source at the well to automatically emit light. The controller may provide these instructions based on any number of triggers. For example, the controller may provide the instructions after a given amount of time has passed such that periodic analyses of the groundwater are performed at various intervals.

In further embodiments, instructions may also be transmitted from a remote system to the groundwater collection and analysis apparatus as well. For example, a remote server may automatically transmit an instruction to a groundwater collection and analysis apparatus at a well to provide data relating to the groundwater at that well. Based on the instruction, the controller of the groundwater collection and analysis apparatus may instruct the light source to emit light into the groundwater collection and analysis apparatus to perform the data collection. The data may then be transmitted back to the remote server for storage. The instructions and data transfer may be performed using any suitable wireless or wired communication protocol. These instructions may also be provided manually by a human operator from a remote location using a device, such as a smartphone, desktop or laptop computer, etc.

Even in embodiments in which a light source is provided at each of the wells, it still may be desirable to deploy a UAV to navigate to some or all of the wells. For example, in some remote locations, a groundwater collection and analysis apparatus may not necessarily have long-range wired or wireless communication capabilities. In such instances, the UAV may be deployed to such locations to provide instructions for the groundwater collection and analysis apparatus to perform an analysis and also for the UAV to collect resulting data. Even if the groundwater collection and analysis apparatus performs its own analyses and stores the data in local data storage, the UAV may be deployed to the location of the groundwater collection and analysis apparatus to retrieve the data from the groundwater collection and analysis apparatus.

Additionally, the UAV may be deployed to the location of a particular groundwater collection and analysis apparatus when it is determined that the groundwater collection and analysis apparatus may be malfunctioning and data is not being received as expected. In instances in which the UAV is deployed to the location of the groundwater collection and analysis apparatus but data is still not able to be retrieved, the UAV may be used for troubleshooting purposes. For example, the UAV may include a camera that is used to capture images and/or video feed of the groundwater collection and analysis apparatus. The UAV may autonomously identify issues with the groundwater collection and analysis apparatus that require maintenance. The UAV may also capture data for troubleshooting using any other types of sensors, wired or wireless communications with the groundwater collection and analysis apparatus, etc. The UAV may then autonomously perform the maintenance and/or may be manually controlled by a human operator to perform the maintenance. As another option, the UAV may provide an indication that maintenance is required and a human may traverse to the location of the well to perform the maintenance.

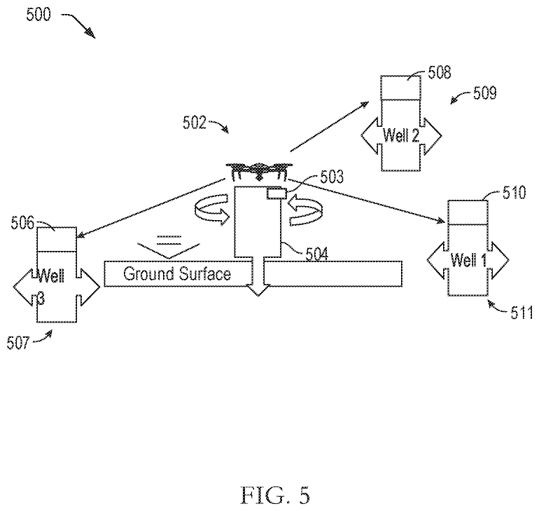

In further embodiments, a docking station may also be provided in the vicinity of one or more of the wells. The docking station may be configured to receive the UAV and rotate in various directions. In this manner, the docketing station may rotate a docked UAV in the direction of a particular groundwater collection and analysis apparatus such that the UAV may then emit light toward that groundwater collection and analysis apparatus. In some instances, it may be difficult for the UAV to perfectly align the light source with the groundwater collection and analysis apparatus while flying. By docking with the docking station, the light source of the UAV may be more accurately aligned with the groundwater collection and analysis apparatus by the rotation of the docking station. The docking station may also include its own light source such that the docking station itself may emit light toward a groundwater collection and analysis apparatus. The docking station may also provide other functionality, such as the ability to charge a UAV that is docked on the docking station, perform wireless and/or wired communications with a UAV, groundwater collection and analysis apparatus, remote system, etc., store data, and/or any other type of functionality. Further details about the docking station are provided with respect to FIG. 5.

While reference is made to the use of a UAV to perform the systems and methods described herein, it should be noted that any other type of vehicle that may traverse to the locations of various wells may also be used. The vehicle may be equipped with any type of autonomous or semi-autonomous functionality and/or may be tele-operated by a user at a remote location. Additionally, while reference is made to a single UAV being deployed to various locations, this is not intended to be limiting and any other number of UAVs and/or other types of vehicles may also be deployed as well.

Turning to the figures, FIG. 1 illustrates a cross-section view of a simplified system 100 for groundwater data collection. In embodiments, the system 100 includes an unmanned aerial vehicle (UAV) 102, a groundwater collection and analysis apparatus 104, and a remote system 120.

The UAV 102 includes a light source 103 that may be used to emit light 105 toward the groundwater collection and analysis apparatus 104. For example, the light source 103 may be a laser that emits light at particular wavelengths towards the groundwater collection and analysis apparatus 104.

The groundwater collection and analysis apparatus 104 may be an apparatus that is provided on and/or within the well 114 and is used to analyze the contents of groundwater 118 included within the well 114 (for example, a fluorescence analysis). The groundwater collection and analysis apparatus 104 may be modular and may be of any varying size and/or shape such that the groundwater collection and analysis apparatus 104 may fit on and/or within the well 114 based on the size and/or shape of the well 114. The groundwater collection and analysis apparatus 104 may also be removable from the well 114 (for example, the groundwater collection and analysis apparatus 104 may be removed from one well and moved to a different well, may be removed for maintenance purposes, or may be removed for any other purpose).

In embodiments, the groundwater collection and analysis apparatus 104 may include a pump 116 and a sample delivery reservoir 110. To analyze the contents of the groundwater 118, a sample of groundwater 118 from the well 114 may be pumped into the sample delivery reservoir 110 by the pump 116.

Any of the data produced by the groundwater collection and analysis apparatus 104 may be wirelessly transmitted to a remote system 120 (for example, a remote server or any other type of system, such as computing device 704, etc.). The data may also be transmitted to the UAV 102 and/or may be stored locally at the groundwater collection and analysis apparatus 104. The UAV 102 and the remote system 120 may also be in wireless communication as well. In this manner, the system 100 may include an Internet of Things (IoT) network in which elements of the system 100 are configured to communicate with one another using wired or wireless transmission protocols.

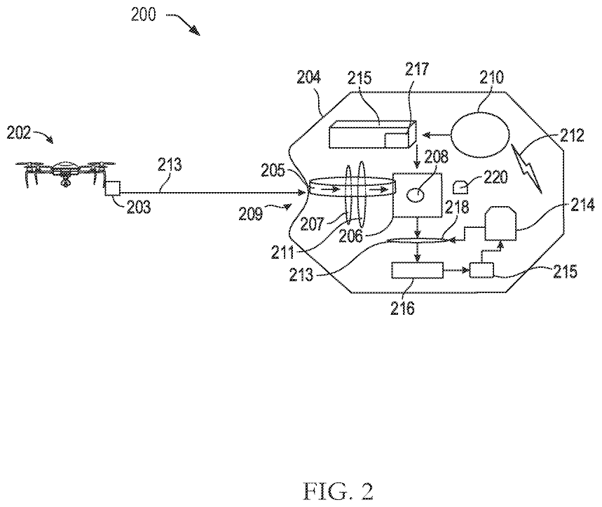

FIG. 2 illustrates another system 200. The system 200 also includes a UAV 202 (which may be the same as UAV 102 or any other UAV described herein) and another example groundwater collection and analysis apparatus 204 (which may be the same as groundwater collection and analysis apparatus 104 or any other groundwater collection and analysis apparatus described herein). The system 200 shows a top-down view of an example groundwater collection and analysis apparatus 204 and provides further details about the components included within the example groundwater collection and analysis apparatus 204. It should be noted that FIG. 2 is merely intended to show examples of components that may be included in the groundwater collection and analysis apparatus 204 and the relative positioning of the components is not intended to be limiting.

Particularly, the groundwater collection and analysis apparatus 204 includes a quartz sampling tube 205 that is configured to receive light 213 that is emitted by the UAV 202 using a light source 203. The groundwater collection and analysis apparatus 204 may also include an optical fiber cable instead of the quartz sampling tube 205 (or in addition to the quartz sampling tube 205). In one particular embodiment, the light source 203 may be a continuous-wave (CW) diode laser with an output power of 1 W at wavelengths ranging between 200 to 800 nm for the detection of groundwater contaminants. However, any other types of light sources may be used that are configured to emit light at any other output powers and wavelengths as well. Additionally, in some instances, the UAV 202 may be configured to dynamically vary the power at which the light is emitted 213, the wavelength(s) of the light 213 that is emitted, or any other parameters of the light 213. For example, wells in specific geographical environments may be subject to certain types of contaminants that are detected by specific wavelength(s). Therefore, the UAV 202 may be configured to selectively emit light 213 at different wavelengths towards a given groundwater collection and analysis apparatus 204 depending on the location of the well at which the groundwater collection and analysis apparatus 204 is provided. In some instances, the UAV 202 may also include multiple different light sources 213 that are each configured to emit light 213 at different power levels, wavelengths, etc. The UAV 202 may also selectively emit light 213 at different wavelengths depending on any other number of factors.

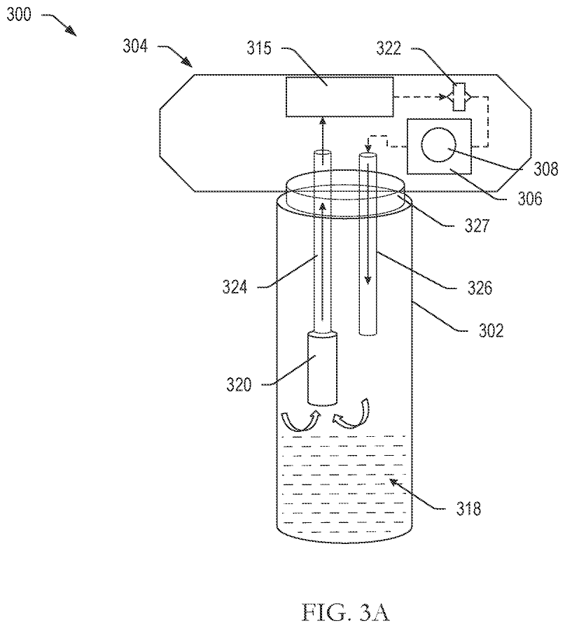

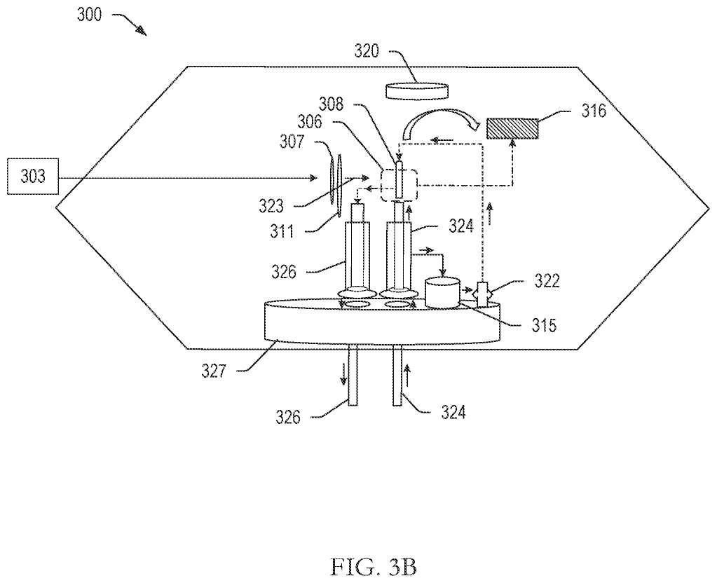

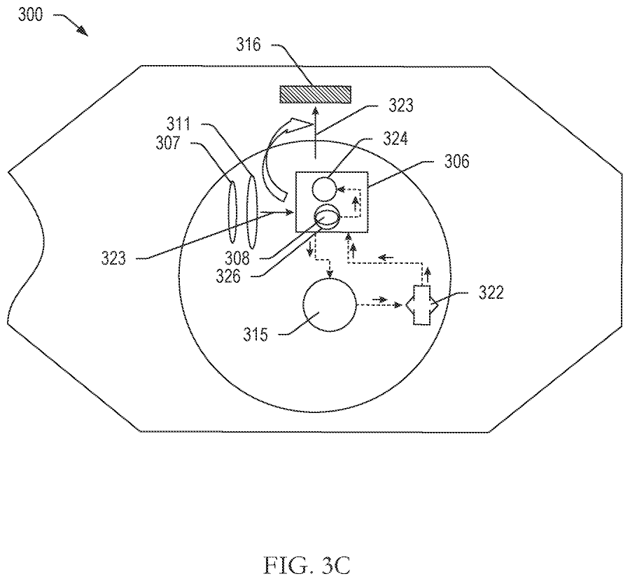

The light 213 that is emitted by the light source 203 of the UAV 202 may be received by the quartz sampling tube 205 via a sampling port 209 provided on the groundwater collection and analysis apparatus 204. That is, the UAV 202 may emit the light 213 via the light source through the sampling port 209 and into the quartz sampling tube 205. The light 213 may pass through the quartz sampling tube 205 towards a cuvette 208. Alternatively, the light 213 may pass through a fiber optic cable (an example is shown as fiber optic cable 323 in FIGS. 3A-3C) towards the cuvette 208 provided in a cuvette holder 206 within the groundwater collection and analysis apparatus 204. That is, the quartz sampling tube 205 and the fiber optical cable may be used as alternatives. However, in some instances, both the quartz sampling tube 205 and the fiber optic cable may be used.

A collimating lens 207 and a wavelength filter 211 may also be provided to ensure that the light signals are flat going into the spectrometer 216 provided in the groundwater collection and analysis apparatus 204.

The cuvette 208 may be a type of holding compartment used to hold a sample of groundwater from the well (and/or a flow through apparatus used to receive a sample of groundwater). When the light 213 received by the groundwater collection and analysis apparatus 204 via the light source 203 reaches the cuvette 208, the light 213 travels through the cuvette and into the spectrometer 216.

To provide a sample of groundwater within the cuvette 208, the sample needs to be transferred from the groundwater located at the bottom of the well upwards into the cuvette 208. This may be accomplished in a number of different ways. As one option shown in FIG. 3, the groundwater collection and analysis apparatus 204 may include one or more pumps 217 that may be used to pump a sample of groundwater into the cuvette 208. In some instances, the one or more pumps 217 may be submersible pumps. Although the one or more pumps 217 are shown as being provided in the reservoir 213, the one or more pumps 217 may also be provided at any other location in the groundwater collection and analysis apparatus 204. FIGS. 3A-3C show further example locations of pumps within the system.

In embodiments in which multiple pumps are used, a first pump may pump water from the groundwater in the well into a groundwater reservoir 215 for temporary storage. A second pump may then pump the water from the groundwater reservoir 215 into the cuvette 208. However, in some instances, only one pump may be used or more than two pumps may be used as well. Additionally, the sample may be pumped directly into the cuvette 208 rather than being pumped into the groundwater reservoir 213 (that is, the groundwater reservoir 213 may not be required). The cuvette 208 may also include a drain tube 230 that is used to return the sample of groundwater back into the well.

Another option is to use a valve 210 to trap vapor that is naturally produced within the well. This vapor may naturally result from changes in barometric pressure within the well (for example, Henry's law states that the amount of dissolved gas in a liquid is directly proportional to its partial pressure above the liquid). For example, the valve 210 may be a one-way check valve, however, other types of valves may be used as well. In this manner, the analysis may involve the use of the vapor sample rather than the use of a liquid groundwater sample. In embodiments, the vapor may also be condensed within the groundwater collection and analysis apparatus 204 such that the vapor sample may be converted into a liquid groundwater sample for analysis. For example, this may be accomplished using a refrigerant-based surface condenser or a capillary condensation system (however, any other methods for converting the vapor sample into a liquid sample may also be used).

In embodiments, the groundwater collection and analysis apparatus 204 may be disinfected using a disinfecting light source 220 that emits ultraviolet light. For example, the disinfecting light source 220 may be a 1.0 mW UV-C LED configured to emit light within a range of wavelengths between 260-270 nm. The UV-C LED may be disabled during groundwater sampling operations but may be periodically activated during times at which analyses are not being performed to disinfect the components of the groundwater collection and analysis apparatus 204. Reference to the UV-C LED is merely exemplary and any other type of disinfecting component may also be used.

Any of the components of the groundwater collection and analysis apparatus 204 may be powered by a power source 212. For example, the power source may be one or more batteries, however, any other types of power sources may also be used. In some instances, solar panels or other types of renewable energy sources may be provided as well. The UAV 202 may also be configured to transport batteries to provide replacements for any old batteries in the groundwater collection and analysis apparatus 204.

Any of the operations of the groundwater collection and analysis apparatus 204 may be controlled by a controller 214, which may be a microprocessor or any other type of device with processing capabilities. In embodiments, the controller 214 may also include associated hardware for performing wireless data transmissions (for example, to receive instructions from the UAV 202 or another device or system, transmit groundwater analysis data to the UAV 202 or another device or system, etc.). In other embodiments, the groundwater collection and analysis apparatus 204 may include a separate transceiver for performing such transmissions. The controller 214 may include any of the elements of the computing device 800.

FIGS. 3A-3C illustrate additional components of a system 300 including a groundwater collection and analysis apparatus 304. The groundwater collection and analysis apparatus 304 may be the same as, or similar to any groundwater collection and analysis apparatus described herein. For example, the groundwater collection and analysis apparatus 304 includes a cuvette 308 and a cuvette holder 306, a reservoir 315, and/or any other components of any groundwater collection and analysis apparatus described herein. FIGS. 3A-3C also shows operation of the pumps (for example, first pump 320 and second pump 322) that may be used to pump water throughout the system 300. It should be noted that FIGS. 3A-3C do not necessarily show all of the components that are included in the groundwater collection and analysis apparatus 304.

Beginning with FIG. 3A, a side view of the system 300 is shown. The side view shows that the first pump 320 is used to pump groundwater 318 from the well 302 towards the groundwater collection and analysis apparatus 304 via extraction tubing 324. For example, the first pump 320 may pump the groundwater 318 into the reservoir 315. However, the first pump 320 may also directly pump the groundwater 318 from the well 302 into the cuvette 308 as well. The first pump 320 may be a submersible pump and may be provided within the groundwater 318. The first pump 320 may also be provided at any other location within the well 302 or outside of the well.

In embodiments, there may be two paths for providing a groundwater sample into the cuvette 308. A first path goes through the extraction tubing 324, directly up to the cuvette 308 fed by the submersible pump. The second path comes up the extraction tubing 324, but has the ability to split off a side stream that may fill the sample reservoir in 315 and feed the pump 322 to the top of the cuvette 308. However, the use of these two paths is optional and a single path may be provided for the groundwater to travel to the cuvette 308 as well.

In embodiments in which the groundwater 318 is pumped by the first pump 320 into the reservoir 315, the second pump 322 may be provided to pump a water sample from the reservoir 315 into the cuvette 308 for analysis. Once an analysis on a groundwater sample has been performed, the sample may be returned back to the well 302 via the return tubing 326.

FIG. 3B shows another side view of the system 300. Particularly, FIG. 3B shows a close-up view of the groundwater collection and analysis apparatus 304, showing some of the additional components of the groundwater collection and analysis apparatus 304 (for example, similar components shown in FIG. 2). For example, FIG. 3B shows that the groundwater collection and analysis apparatus 304 may include a spectrometer 316, a collimating lens 307 and a wavelength filter 311, a disinfecting light source 320, etc.

FIG. 3C shows a top-down view of some of the components of the system 300 shown in FIGS. 3A-3B.

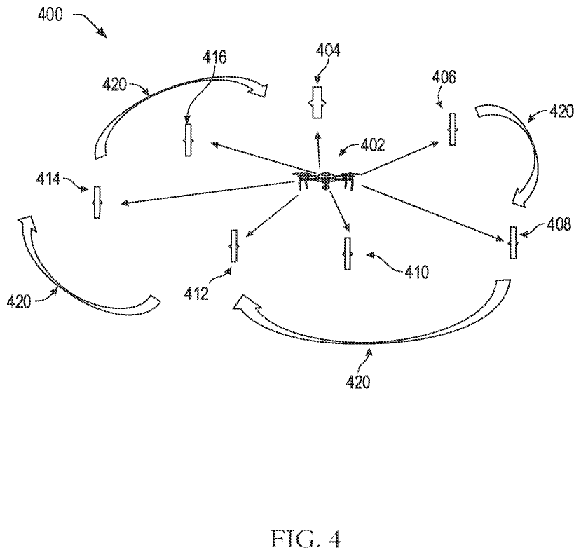

FIG. 4 illustrates another system 400. Particularly, FIG. 4 illustrates that the UAV 402 (which may be the same as UAV 202 or any other UAV described herein) may navigate between various wells at different locations to initiate groundwater data analysis at the wells and/or collect resulting data. For example, FIG. 4 shows the UAV 402 navigating a path including a first waypoint 404, second waypoint 406, third waypoint 408, fourth waypoint 410, fifth waypoint 412, sixth waypoint 414, and seventh waypoint 416.

Each of the waypoints may include a well and a groundwater collection and analysis apparatus may be provided on and/or within each of the wells. The UAV 402 may navigate to each of the waypoints and emit light toward the groundwater collection and analysis apparatuses located at each of the waypoints. The light may interact with each of the groundwater collection and analysis apparatus in a manner described herein such that an analysis of the contents of the groundwater in each of the wells may be analyzed and data about the contents may be obtained. This data may be transmitted to the UAV 402 from each of the groundwater collection and analysis apparatuses. The UAV 402 may store this data in memory and/or may transmit the data to a remote system (such as the remote system 120 shown in FIG. 1) for storage.

In some cases, the path 420 may be a preset path that the UAV 402 periodically navigates to collect data from the wells at each of the waypoints. The UAV 402 may have autonomous capabilities such that the UAV 402 may automatically navigate the path 420 and perform data collection from each of the waypoints. However, in other cases, the navigation of the UAV 402 may also be remotely controlled by a user as well (for example, a user may tele-operate the UAV 402).

Additionally, the path of the 420 may not necessarily be fixed and may vary depending on a number of factors. For example, it may be desirable to obtain data from certain locations at different points in time so the path 420 may be adjusted such that the UAV 402 only navigates to some of the waypoints. As new groundwater collection and analysis apparatuses are added to different wells and groundwater collection and analysis apparatuses are removed from wells at existing waypoints, the path 420 may be adjusted such that the UAV 402 only navigates to wells including groundwater collection and analysis apparatuses. The path 402 may also be adjusted in for any other reasons. Further, the UAV 40 sampling port 2092 may also automatically or manually navigate to only a single well, rather than navigating the path 420 to multiple waypoints as well.

FIG. 5 illustrates another system 500. The system 500 may also include a UAV 502 and one or more groundwater collection and analysis apparatuses (for example, groundwater collection and analysis apparatus 506, groundwater collection and analysis apparatuses 508, groundwater collection and analysis apparatuses 510, etc.) provided on and/or within each of one or more wells (for example, well 507, well 509, well 511, etc.). The UAV 502 and groundwater collection and analysis apparatuses shown in FIG. 5 may also be the same as any other UAVs or groundwater collection and analysis apparatuses described herein.

In addition to the UAV 502 and the one or more groundwater collection and analysis apparatuses, the system 500 also includes a docking station 504. The docking station 504 may be configured to receive the UAV 502 and rotate in various directions. In this manner, the docketing station 504 may rotate a docked UAV in the direction of a particular groundwater collection and analysis apparatus such that the UAV 502 may then emit light towards that groundwater collection and analysis apparatus. In some instances, it may be difficult for the UAV 502 to perfectly align the light source with the groundwater collection and analysis apparatus while flying. By docking with the docking station 504, the light source of the UAV 502 may be more accurately aligned with the groundwater collection and analysis apparatus by the rotation of the docking station 504. The alignment may be performed in any suitable manner (for example, using a camera and/or other type of sensors of the UAV 502 and/or the docking station 504, wireless communications between the UAV 502 and the docking station 504 using any wireless communication protocol, mechanical mechanisms for guiding the UAV 502 into the docking station 504 and locking the UAV 502 into the docking station 504, etc.

The docking station 504 may be provided remotely from the one or more wells but may be sufficiently proximate to the one or more wells such that light emitted by the light source of the UAV 502 may be received by the groundwater collection and analysis apparatuses when the UAV 502 is located at the docking station 504. In some instances, it may be difficult for the UAV 502 to properly align the light source mounted to the UAV 502 such that light emitted by the UAV 502 is received by the groundwater collection and analysis at the proper location for analysis of the content of the groundwater (for example, the sampling port 209).

In embodiments, the docking station 504 may also include its own light source 503. In such embodiments, the docking station 504 may be able to emit light towards the groundwater collection and analysis apparatuses in a similar manner that the light source from the UAV 502 may emit light towards the groundwater collection and analysis apparatuses. In this manner, the groundwater data collection may be performed without requiring the UAV 502. For example, the docking station 504 may rotate towards the location of each of the groundwater collection and analysis apparatuses, emit light towards each of the groundwater collection and analysis apparatuses, and receive any resulting data produced by the groundwater collection and analysis apparatuses. The docking station 504 may store the data in memory or may transmit the data to a remote system (such as remote system 120 in FIG. 1). The data may also be directly transmitted from the groundwater collection and analysis apparatuses to the remote system and the docking station 504 may only initiate the data collection by emitting the light towards the groundwater collection and analysis apparatuses. In further cases, the UAV 502 may still navigate towards the location of the one or more wells but may not dock on the docking station 504. Instead, the UAV 502 may wirelessly transmit an instruction to the docking station 504 and the docking station 504 may then emit light towards a groundwater collection and analysis apparatus.

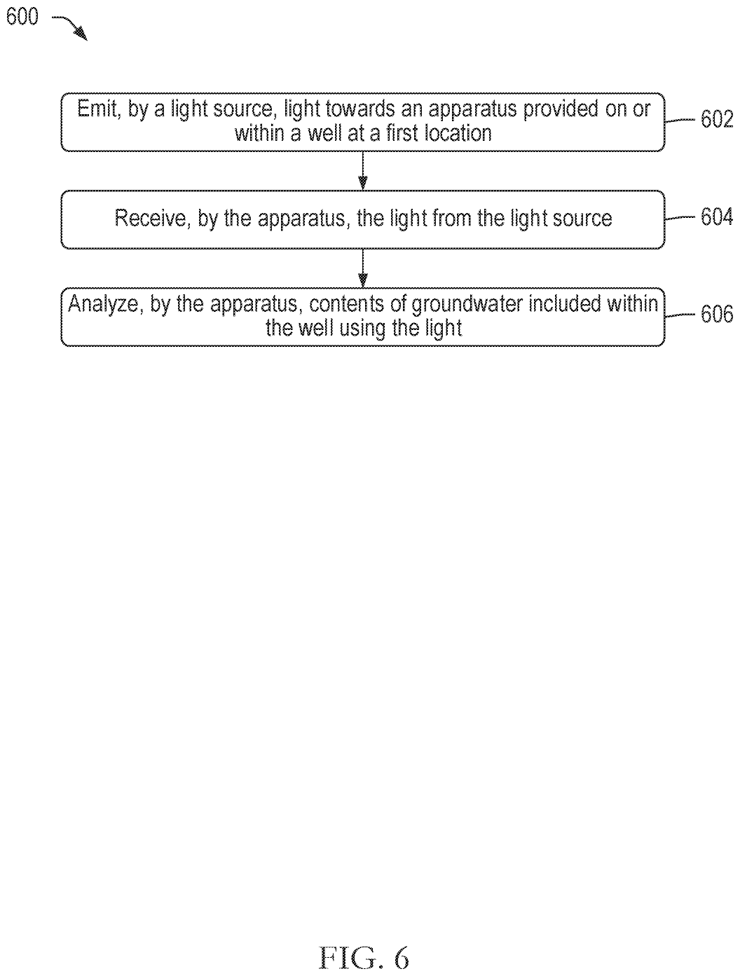

FIG. 6 depicts an example method 600 for training a machine learning model using compartmentalization. Some or all of the blocks of the process flows or methods in this disclosure may be performed in a distributed manner across any number of devices or systems (for example, UAVs 102, 202, 402, 502, 706, groundwater collection and analysis apparatuses 104, 204, 506, 508, 510, 708, computing device 704, computing device 800, etc.). The operations of the method 600 may be optional and may be performed in a different order.

At block 602 of the method 600, computer-executable instructions stored on a memory of a system or device, such as, user device 501, computing device 504, computing device 600, etc., may be executed to emit, by a light source, light towards an apparatus provided on or within a well at a first location.

At block 604 of the method 600, computer-executable instructions stored on a memory of a system or device, such as, user device 501, computing device 504, computing device 600, etc., may be executed to receive, by the apparatus, the light from the light source.

At block 606 of the method 600, computer-executable instructions stored on a memory of a system or device, such as, user device 501, computing device 504, computing device 600, etc., may be executed to analyze, by the apparatus, contents of groundwater included within the well using the light.

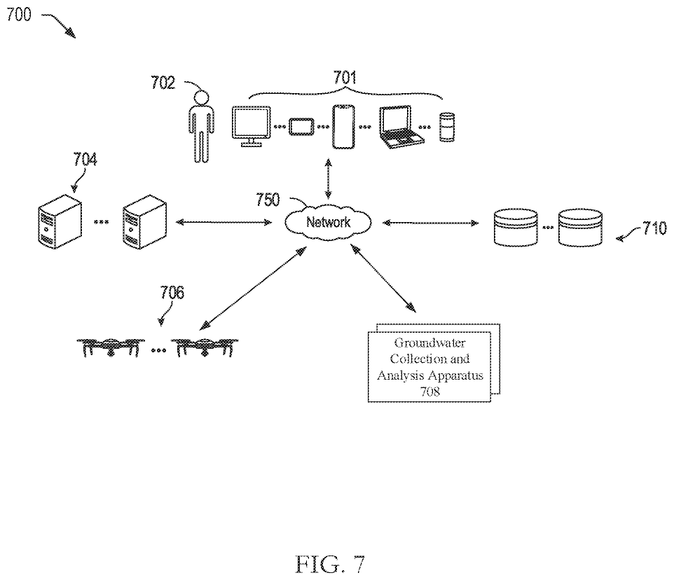

FIG. 7 is an example system 700 for generating distinct images using a generative model. In one or more embodiments, the system may include one or more user devices 701 (which may be associated with one or more users 702), one or more computing devices 704, one or more UAVs 706, one or more groundwater collection and analysis apparatuses 708, and/or one or more databases 710. However, these components of the system 700 are merely exemplary and are not intended to be limiting in any way. For simplicity, reference may be made hereinafter to a user device 701, computing device 704, UAV 706, groundwater collection and analysis apparatus 708, database 710, etc., however, this is not intended to be limiting and may still refer to any number of such elements.

The user device 701 may be any type of device, such as a smartphone, desktop computer, laptop computer, tablet, smart television (for example, a television with Internet connectivity, the capability to install applications, etc.), and/or any other type of device. The user device 701 may allow a user 702 to interact with any of the systems, devices, etc. to perform any number of different types of actions, transmitting instructions to a UAV 706 to navigate to collect data from a groundwater collection and analysis apparatus 708, performing teleoperation controls of a UAV 706, and/or any other type of functionality described herein or otherwise.

The user device 701 may also include an application that may allow the user 702 to view groundwater analysis data obtained from the groundwater collection and analysis apparatus 708. For example, the user 702 may be able to view information about the contents of the groundwater of a particular well associated with the groundwater collection and analysis apparatus 708 (such as contaminants included in the groundwater or any other type of information). The user 702 may also be able to perform any of the aforementioned operations using the application as well.

The computing device 704 may be any type of device (such as a local or remote server for example) used to perform any of the processing described herein. For example, the computing device 704 may be the same as, or similar to, the remote system 120 shown in FIG. 1. The computing device 704 may receive and/or store (for example, via the database 710) data obtained from the groundwater collection and analysis apparatus 708. The computing device 708 may receive this data directly from the groundwater collection and analysis apparatus 708, from the UAV 706, and/or any other device, system, etc. In embodiments, the computing device 704 may also transmit instructions to the UAV 706 to navigate to the groundwater collection and analysis apparatus 708 and obtain data or may transmit instructions to the groundwater collection and analysis apparatus 708 to perform a groundwater analysis and transmit data back to the computing device 708.

The UAV 706 may be the same as, or similar to any of the UAVs described herein (for example, UAV 102, 202, 402, 502, etc.). The UAV 706 may be an autonomous UAV 706 and may autonomously navigate a path to different groundwater collection and analysis apparatuses 708 to emit light toward the groundwater collection and analysis apparatuses 708 to produce groundwater sample data from wells associated with the groundwater collection and analysis apparatuses 708 and/or collect the data from the groundwater collection and analysis apparatuses 708. The UAV 706 may also be manually operated be a remote operation, such as through tele-operation commands from a remote system.

As aforementioned, the system 700 may not necessarily be limited to the use of only a UAV 706. Any other type of vehicle that may traverse to the locations of various wells may also be used. Additionally, while reference is made to a single UAV 706 being deployed to various locations, this is not intended to be limiting and any other number of UAVs 706 and/or other types of vehicles may also be deployed as well. In some instances, multiple vehicles of different types may be deployed to different locations depending on the terrain of the location or other factors.

The groundwater collection and analysis apparatus 708 may be the same as, or similar to, any of the groundwater collection and analysis apparatuses described herein (for example, groundwater collection and analysis apparatuses 104, 204, 506, 508, 510, etc.).

In one or more embodiments, any of the elements of the system 700 (for example, one or more user devices 701, one or more computing devices 704, one or more UAVs 706, one or more groundwater collection and analysis apparatuses 708, one or more databases 710, and/or any other element described with respect to FIG. 7 or otherwise) may be configured to communicate via a communications network 750. The communications network 750 may include, but not limited to, any one of a combination of different types of suitable communications networks such as, for example, broadcasting networks, cable networks, public networks (e.g., the Internet), private networks, wireless networks, cellular networks, or any other suitable private and/or public networks. Further, the communications network 750 may have any suitable communication range associated therewith and may include, for example, global networks (e.g., the Internet), metropolitan area networks (MANs), wide area networks (WANs), local area networks (LANs), or personal area networks (PANs). In addition, communications network 750 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, white space communication mediums, ultra-high frequency communication mediums, satellite communication mediums, or any combination thereof.

In this manner, the elements of the system 700 may form an IoT network such that data may be transmitted between various elements of the system 700. For example, the groundwater analysis data may be transmitted between the user device 701, computing device 704, UAV 706, and/or groundwater collection and analysis apparatus 708.

Finally, any of the elements (for example, one or more user devices 701, one or more computing devices 704, one or more UAVs 706, one or more groundwater collection and analysis apparatuses 708, and/or one or more databases 710) of the system 700 may include any of the elements of the computing device 800 as well (such as the processor 802, memory 804, etc.).

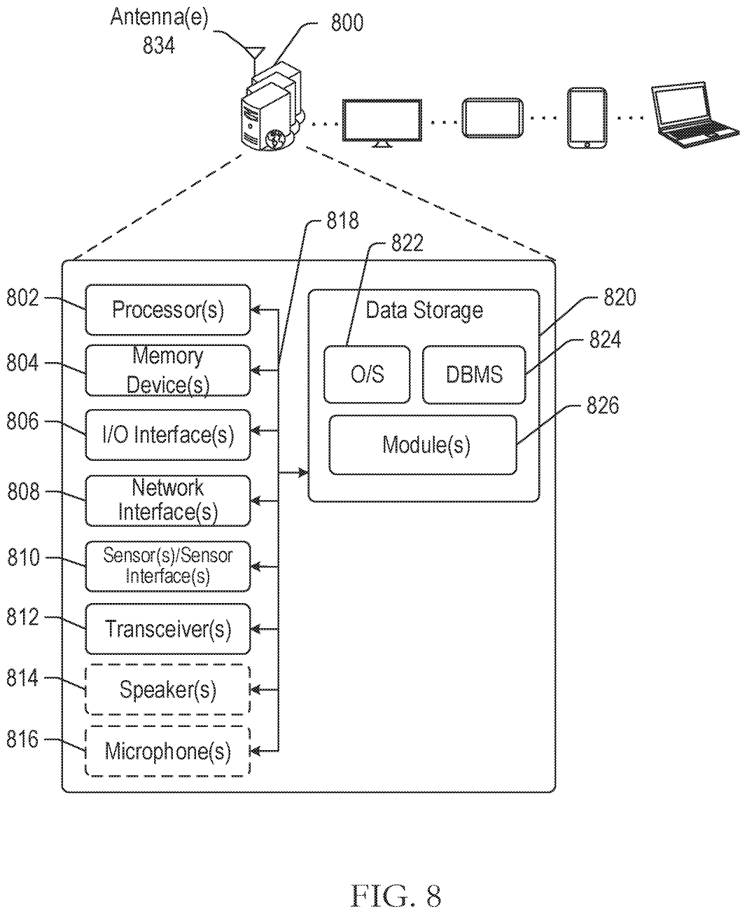

FIG. 8 is a schematic block diagram of an illustrative computing device 800 in accordance with one or more example embodiments of the disclosure. The computing device 800 may include any suitable computing device capable of receiving and/or generating data including, but not limited to, a user device such as a smartphone, tablet, e-reader, wearable device, or the like; a desktop computer; a laptop computer; a content streaming device; a set-top box; or the like. The computing device 800 may correspond to an illustrative device configuration for the devices of FIGS. 1-7 (for example, UAVs 102, 202, 402, 502, 706, groundwater collection and analysis apparatuses 104, 204, 506, 508, 510, 708, computing device 704, computing device 800, etc.).

The computing device 800 may be configured to communicate via one or more networks with one or more servers, search engines, user devices, or the like. In some embodiments, a single remote server or single group of remote servers may be configured to perform more than one type of content rating and/or machine learning functionality.

Example network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks. Further, such network(s) may have any suitable communication range associated therewith and may include, for example, global networks (e.g., the Internet), metropolitan area networks (MANs), wide area networks (WANs), local area networks (LANs), or personal area networks (PANs). In addition, such network(s) may include communication links and associated networking devices (e.g., link-layer switches, routers, etc.) for transmitting network traffic over any suitable type of medium including, but not limited to, coaxial cable, twisted-pair wire (e.g., twisted-pair copper wire), optical fiber, a hybrid fiber-coaxial (HFC) medium, a microwave medium, a radio frequency communication medium, a satellite communication medium, or any combination thereof.

In an illustrative configuration, the computing device 800 may include one or more processors (processor(s)) 802, one or more memory devices 804 (generically referred to herein as memory 804), one or more input/output (I/O) interface(s) 806, one or more network interface(s) 808, one or more sensors or sensor interface(s) 810, one or more transceivers 812, one or more optional speakers 814, one or more optional microphones 816, and data storage 820. The computing device 800 may further include one or more buses 818 that functionally couple various components of the computing device 800. The computing device 800 may further include one or more antenna(e) 834 that may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals, a Near Field Communication (NFC) antenna for transmitting or receiving NFC signals, and so forth. These various components will be described in more detail hereinafter.

The bus(es) 818 may include at least one of a system bus, a memory bus, an address bus, or a message bus, and may permit exchange of information (e.g., data (including computer-executable code), signaling, etc.) between various components of the computing device 800. The bus(es) 818 may include, without limitation, a memory bus or a memory controller, a peripheral bus, an accelerated graphics port, and so forth. The bus(es) 818 may be associated with any suitable bus architecture including, without limitation, an Industry Standard Architecture (ISA), a Micro Channel Architecture (MCA), an Enhanced ISA (EISA), a Video Electronics Standards Association (VESA) architecture, an Accelerated Graphics Port (AGP) architecture, a Peripheral Component Interconnects (PCI) architecture, a PCI-Express architecture, a Personal Computer Memory Card International Association (PCMCIA) architecture, a Universal Serial Bus (USB) architecture, and so forth.

The memory 804 of the computing device 800 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In certain example embodiments, volatile memory may enable faster read/write access than non-volatile memory. However, in certain other example embodiments, certain types of non-volatile memory (e.g., FRAM) may enable faster read/write access than certain types of volatile memory.

In various implementations, the memory 804 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth. The memory 804 may include main memory as well as various forms of cache memory such as instruction cache(s), data cache(s), translation lookaside buffer(s) (TLBs), and so forth. Further, cache memory such as a data cache may be a multi-level cache organized as a hierarchy of one or more cache levels (L1, L2, etc.).

The data storage 820 may include removable storage and/or non-removable storage including, but not limited to, magnetic storage, optical disk storage, and/or tape storage. The data storage 820 may provide non-volatile storage of computer-executable instructions and other data. The memory 804 and the data storage 820, removable and/or non-removable, are examples of computer-readable storage media (CRSM) as that term is used herein.

The data storage 820 may store computer-executable code, instructions, or the like that may be loadable into the memory 804 and executable by the processor(s) 802 to cause the processor(s) 802 to perform or initiate various operations. The data storage 820 may additionally store data that may be copied to memory 804 for use by the processor(s) 802 during the execution of the computer-executable instructions. Moreover, output data generated as a result of execution of the computer-executable instructions by the processor(s) 802 may be stored initially in memory 804, and may ultimately be copied to data storage 820 for non-volatile storage.

More specifically, the data storage 820 may store one or more operating systems (O/S) 822; one or more database management systems (DBMS) 824; and one or more program module(s), applications, engines, computer-executable code, scripts, or the like such as, for example, one or more module(s) 826. Any of the components depicted as being stored in data storage 820 may include any combination of software, firmware, and/or hardware. The software and/or firmware may include computer-executable code, instructions, or the like that may be loaded into the memory 804 for execution by one or more of the processor(s) 802. Any of the components depicted as being stored in data storage 820 may support functionality described in reference to correspondingly named components earlier in this disclosure.

The data storage 820 may further store various types of data utilized by components of the computing device 800. Any data storaged in the data storage 820 may be loaded into the memory 804 for use by the processor(s) 802 in executing computer-executable code. In addition, any data depicted as being stored in the data storage 820 may potentially be stored in one or more datastore(s) and may be accessed via the DBMS 824 and loaded in the memory 804 for use by the processor(s) 802 in executing computer-executable code. The datastore(s) may include, but are not limited to, databases (e.g., relational, object-oriented, etc.), file systems, flat files, distributed datastores in which data is stored on more than one node of a computer network, peer-to-peer network datastores, or the like. In FIG. 8, the datastore(s) may include, for example, purchase history information, user action information, user profile information, a database linking search queries and user actions, and other information.

The processor(s) 802 may be configured to access the memory 804 and execute computer-executable instructions loaded therein. For example, the processor(s) 802 may be configured to execute computer-executable instructions of the various program module(s), applications, engines, or the like of the computing device 800 to cause or facilitate various operations to be performed in accordance with one or more embodiments of the disclosure. The processor(s) 802 may include any suitable processing unit capable of accepting data as input, processing the input data in accordance with stored computer-executable instructions, and generating output data. The processor(s) 802 may include any type of suitable processing unit including, but not limited to, a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. Further, the processor(s) 802 may have any suitable microarchitecture design that includes any number of constituent components such as, for example, registers, multiplexers, arithmetic logic units, cache controllers for controlling read/write operations to cache memory, branch predictors, or the like. The microarchitecture design of the processor(s) 802 may be capable of supporting any of a variety of instruction sets.

Referring now to functionality supported by the various program module(s) depicted in FIG. 8, the module(s) 826 may include computer-executable instructions, code, or the like that responsive to execution by one or more of the processor(s) 802 may perform any functions of any of the systems and/or devices described herein (e.g., a UAV, user device, computing device, docking station, groundwater collection and analysis apparatus, etc.

Referring now to other illustrative components depicted as being stored in the data storage 820, the O/S 822 may be loaded from the data storage 820 into the memory 804 and may provide an interface between other application software executing on the computing device 800 and hardware resources of the computing device 800. More specifically, the O/S 822 may include a set of computer-executable instructions for managing hardware resources of the computing device 800 and for providing common services to other application programs (e.g., managing memory allocation among various application programs). In certain example embodiments, the O/S 822 may control execution of the other program module(s) to dynamically enhance characters for content rendering. The O/S 822 may include any operating system now known or which may be developed in the future including, but not limited to, any server operating system, any mainframe operating system, or any other proprietary or non-proprietary operating system.

The DBMS 824 may be loaded into the memory 804 and may support functionality for accessing, retrieving, storing, and/or manipulating data storaged in the memory 804 and/or data storaged in the data storage 820. The DBMS 824 may use any of a variety of database models (e.g., relational model, object model, etc.) and may support any of a variety of query languages. The DBMS 824 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like. In those example embodiments in which the computing device 800 is a user device, the DBMS 824 may be any suitable light-weight DBMS optimized for performance on a user device.

Referring now to other illustrative components of the computing device 800, the input/output (I/O) interface(s) 806 may facilitate the receipt of input information by the computing device 800 from one or more I/O devices as well as the output of information from the computing device 800 to the one or more I/O devices. The I/O devices may include any of a variety of components such as a display or display screen having a touch surface or touchscreen; an audio output device for producing sound, such as a speaker; an audio capture device, such as a microphone; an image and/or video capture device, such as a camera; a haptic unit; and so forth. Any of these components may be integrated into the computing device 800 or may be separate. The I/O devices may further include, for example, any number of peripheral devices such as data storage devices, printing devices, and so forth.

The I/O interface(s) 806 may also include an interface for an external peripheral device connection such as universal serial bus (USB), FireWire, Thunderbolt, Ethernet port or other connection protocol that may connect to one or more networks. The I/O interface(s) 806 may also include a connection to one or more of the antenna(e) 834 to connect to one or more networks via a wireless local area network (WLAN) (such as Wi-Fi) radio, Bluetooth, ZigBee, and/or a wireless network radio, such as a radio capable of communication with a wireless communication network such as a Long Term Evolution (LTE) network, WiMAX network, 3G network, ZigBee network, etc.

The computing device 800 may further include one or more network interface(s) 808 via which the computing device 800 may communicate with any of a variety of other systems, platforms, networks, devices, and so forth. The network interface(s) 808 may enable communication, for example, with one or more wireless routers, one or more host servers, one or more web servers, and the like via one or more of networks.

The antenna(e) 834 may include any suitable type of antenna depending, for example, on the communications protocols used to transmit or receive signals via the antenna(e) 834. Non-limiting examples of suitable antennas may include directional antennas, non-directional antennas, dipole antennas, folded dipole antennas, patch antennas, multiple-input multiple-output (MIMO) antennas, or the like. The antenna(e) 834 may be communicatively coupled to one or more transceivers 812 or radio components to which or from which signals may be transmitted or received.

As previously described, the antenna(e) 834 may include a cellular antenna configured to transmit or receive signals in accordance with established standards and protocols, such as Global System for Mobile Communications (GSM), 3G standards (e.g., Universal Mobile Telecommunications System (UMTS), Wideband Code Division Multiple Access (W-CDMA), CDMA2000, etc.), 4G standards (e.g., Long-Term Evolution (LTE), WiMax, etc.), direct satellite communications, or the like.

The antenna(e) 834 may additionally, or alternatively, include a Wi-Fi antenna configured to transmit or receive signals in accordance with established standards and protocols, such as the IEEE 802.11 family of standards, including via 2.4 GHz channels (e.g., 802.11b, 802.11g, 802.11n), 5 GHz channels (e.g., 802.11n, 802.11ac), or 60 GHz channels (e.g., 802.11ad). In alternative example embodiments, the antenna(e) 834 may be configured to transmit or receive radio frequency signals within any suitable frequency range forming part of the unlicensed portion of the radio spectrum.

The antenna(e) 834 may additionally, or alternatively, include a GNSS antenna configured to receive GNSS signals from three or more GNSS satellites carrying time-position information to triangulate a position therefrom. Such a GNSS antenna may be configured to receive GNSS signals from any current or planned GNSS such as, for example, the Global Positioning System (GPS), the GLONASS System, the Compass Navigation System, the Galileo System, or the Indian Regional Navigational System.

The transceiver(s) 812 may include any suitable radio component(s) for—in cooperation with the antenna(e) 834—transmitting or receiving radio frequency (RF) signals in the bandwidth and/or channels corresponding to the communications protocols utilized by the computing device 800 to communicate with other devices. The transceiver(s) 812 may include hardware, software, and/or firmware for modulating, transmitting, or receiving—potentially in cooperation with any of antenna(e) 834—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 812 may further include hardware, firmware, or software for receiving GNSS signals. The transceiver(s) 812 may include any known receiver and baseband suitable for communicating via the communications protocols utilized by the computing device 800. The transceiver(s) 812 may further include a low noise amplifier (LNA), additional signal amplifiers, an analog-to-digital (A/D) converter, one or more buffers, a digital baseband, or the like.

The sensor(s)/sensor interface(s) 810 may include or may be capable of interfacing with any suitable type of sensing device such as, for example, inertial sensors, force sensors, thermal sensors, and so forth. Example types of inertial sensors may include accelerometers (e.g., MEMS-based accelerometers), gyroscopes, and so forth.

The optional speaker(s) 814 may be any device configured to generate audible sound. The optional microphone(s) 816 may be any device configured to receive analog sound input or voice data.

It should be appreciated that the program module(s), applications, computer-executable instructions, code, or the like depicted in FIG. 8 as being stored in the data storage 820 are merely illustrative and not exhaustive and that processing described as being supported by any particular module may alternatively be distributed across multiple module(s) or performed by a different module. In addition, various program module(s), script(s), plug-in(s), Application Programming Interface(s) (API(s)), or any other suitable computer-executable code hosted locally on the computing device 800, and/or hosted on other computing device(s) accessible via one or more networks, may be provided to support functionality provided by the program module(s), applications, or computer-executable code depicted in FIG. 8 and/or additional or alternate functionality. Further, functionality may be modularized differently such that processing described as being supported collectively by the collection of program module(s) depicted in FIG. 8 may be performed by a fewer or greater number of module(s), or functionality described as being supported by any particular module may be supported, at least in part, by another module. In addition, program module(s) that support the functionality described herein may form part of one or more applications executable across any number of systems or devices in accordance with any suitable computing model such as, for example, a client-server model, a peer-to-peer model, and so forth. In addition, any of the functionality described as being supported by any of the program module(s) depicted in FIG. 8 may be implemented, at least partially, in hardware and/or firmware across any number of devices.

It should further be appreciated that the computing device 800 may include alternate and/or additional hardware, software, or firmware components beyond those described or depicted without departing from the scope of the disclosure. More particularly, it should be appreciated that software, firmware, or hardware components depicted as forming part of the computing device 800 are merely illustrative and that some components may not be present or additional components may be provided in various embodiments. While various illustrative program module(s) have been depicted and described as software module(s) stored in data storage 820, it should be appreciated that functionality described as being supported by the program module(s) may be enabled by any combination of hardware, software, and/or firmware. It should further be appreciated that each of the above-mentioned module(s) may, in various embodiments, represent a logical partitioning of supported functionality. This logical partitioning is depicted for ease of explanation of the functionality and may not be representative of the structure of software, hardware, and/or firmware for implementing the functionality. Accordingly, it should be appreciated that functionality described as being provided by a particular module may, in various embodiments, be provided at least in part by one or more other module(s). Further, one or more depicted module(s) may not be present in certain embodiments, while in other embodiments, additional module(s) not depicted may be present and may support at least a portion of the described functionality and/or additional functionality. Moreover, while certain module(s) may be depicted and described as sub-module(s) of another module, in certain embodiments, such module(s) may be provided as independent module(s) or as sub-module(s) of other module(s).

Program module(s), applications, or the like disclosed herein may include one or more software components including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

That which is claimed is:

1. A system comprising:
a light source configured to emit light; and
an apparatus provided on or within a well, wherein the apparatus is configured to analyze contents of groundwater included within the well using the light, and wherein the well is located at a first location.

2. The system of claim 1, wherein the light source is provided on an unmanned aerial vehicle (UAV), wherein the UAV is configured to navigate to a location of the well and emit the light towards the apparatus.

3. The system of claim 2, wherein the apparatus further comprises a controller configured to transmit data resulting from analyzing the contents of the groundwater to the UAV or a remote system.

4. The system of claim 2, wherein the system comprises a plurality of wells and a plurality of apparatuses, and wherein the UAV is configured to autonomously navigate a path between the plurality of wells.

5. The system of claim 2, further comprising:
a docking station configured to receive the UAV, wherein the docking station is located at a second location, wherein the docking station is configured to rotate the UAV such that the light source is facing in a direction towards the apparatus at the first location.

6. The system of claim 1, wherein the light source is a light emitting diode that is provided on or within the well.

7. The system of claim 1, wherein the apparatus further comprises:
a quartz sampling tube configured to receive the light from the light source;
a cuvette configured to hold a sample of the groundwater from the well;
a fiber optic cable configured to direct the light towards the cuvette; and
a spectrometer configured to receive the light from the cuvette and analyze the light to determine the contents of the groundwater.

8. The system of claim 7, wherein the apparatus further comprises:
a pump configured to pump the groundwater from the well into the cuvette.

9. The system of claim 7, wherein the apparatus further comprises:
a one-way valve configured to receive vapor produced by the groundwater, wherein the vapor is temporarily stored in the cuvette.

10. A method comprising:
emitting, by a light source, light towards an apparatus provided on or within a well at a first location;
receiving, by the apparatus, the light from the light source; and
analyzing, by the apparatus, contents of groundwater included within the well using the light.

11. The method of claim 10, wherein the light source is provided on an unmanned aerial vehicle (UAV), and wherein the method further comprises:
navigating, by the UAV, to a location of the well and emit the light towards the apparatus.

12. The method of claim 11, further comprising:
transmitting, by a controller of the apparatus, data resulting from analyzing the contents of the groundwater to the UAV or a remote system.

13. The method of claim 11, further comprising:
autonomously navigating, by the UAV, between a plurality wells and a plurality of apparatuses.

14. The method of claim 11, further comprising:
receiving, by a docking station at a second location, the UAV; and
rotating, by the docking station, the UAV such that the light source is facing in a direction towards the apparatus at the first location.

15. The method of claim 10, wherein the light source is a light emitting diode that is provided on or within the well.

16. The method of claim 10, further comprising:
receiving, by a quartz sampling tube of the apparatus, the light from the light source;
receiving, by a cuvette, a sample of the groundwater from the well;
directing, by a fiber optic cable, the light towards the cuvette;
receiving, by a spectrometer, the light from the cuvette; and
analyzing, using the spectrometer, the light to determine the contents of the groundwater.

17. The method of claim 16, further comprising:
pumping, by a pump of the apparatus, the groundwater from the well into the cuvette.

18. The method of claim 16, further comprising:
a one-way valve configured to receive vapor produced by the groundwater, wherein the vapor is temporarily stored in the cuvette.

19. A system comprising:
a UAV including a light source configured to emit light; and
an apparatus provided on or within a well, wherein the apparatus is configured to analyze contents of groundwater included within the well using the light, and wherein the well is located at a first location.

20. The system of claim 19, wherein the apparatus further comprises:
a quartz sampling tube configured to receive the light from the light source;
a cuvette configured to hold a sample of the groundwater from the well;
a fiber optic cable configured to direct the light towards the cuvette; and
a spectrometer configured to receive the light from the cuvette and analyze the light to determine the contents of the groundwater.
